(19) United States
(12) Patent Application Publication
(10) Pub. No.: US 2025/0276105 A1
(43) Pub. Date: Sep. 4, 2025

(54) DEVICE FOR DISPENSING VOLATILE SUBSTANCES

(71) Applicant: UWM Research Foundation, Inc., Milwaukee, WI (US)

(72) Inventors: Krishna M. Pillai, Milwaukee, WI (US); Abul Borkot Md Rafiqul Hasan, Milwaukee, WI (US)

(21) Appl. No.: 19/070,020

(22) Filed: Mar. 4, 2025

Related U.S. Application Data
(60) Provisional application No. 63/561,142, filed on Mar. 4, 2024.

(51) Int. Cl. A61L 9/12; A01M 29/12

(52) U.S. Cl. CPC A61L 9/127; A01M 29/12; A61L 9/122; A61L 2209/111; A61L 2209/134

(57) ABSTRACT

A device for dispensing volatile substances to a surrounding environment includes a reservoir configured to hold a multi-component liquid mixture of non-volatile substances and volatile substances, a wick configured to be in selective communication with the liquid mixture in reservoir, an air inducing device that provides an airflow over the wick to release the volatile substances to a surrounding environment, and a rotating assembly coupled to the wick, the rotating assembly configured to rotate the wick to periodically replenish the wick with the liquid mixture prior to introducing the wick to the airflow.

### CROSS-REFERENCE TO RELATED APPLICATIONS

This application claims priority to U.S. Provisional Patent Application No. 63/561,142, which was filed on Mar. 4, 2024 and entitled “Device for Dispensing Volatile Substances,” the contents of which is hereby incorporated by reference in its entirety.

### FIELD

The present disclosure relates to devices that dispense a volatile substance, including, but not limited to fragrances, insect repellent, or the like. More particularly, this disclosure is directed to a device that includes an assembly configured to rotate at least one wick between contact with a liquid mixture that includes the volatile substance and is absorbed by the wick, and an airflow generated by the device to disperse the volatile substance from the wick.

### BACKGROUND

Dispersed volatile substances, like incense or insect repellent, is generally a liquid mixture that consists of a volatile liquid component mixed with a non-volatile (or less-volatile) liquid base. Current wick-based plug-in devices used for the spreading of volatile substances, like incense or insect repellent, in a local environment suffer from a decline in performance over time due to a reduction in the delivery of the volatile substances. This decrease in volatile substance distribution generally occurs because over time, the non-volatile (or less-volatile) components of the liquid mixture build up and block the wick, decreasing the effectiveness of the wick to disperse the desired volatile component of the liquid mixture.

The discussion above is merely provided for general background information and is not intended to be used as an aid in determining the scope of the claimed subject matter.

### SUMMARY

In one aspect, the disclosure is directed to a device for dispensing volatile substances to a surrounding environment, the device including a reservoir configured to hold a multi-component liquid mixture of non-volatile substances and volatile substances; a wick configured to be in selective communication with the liquid mixture in reservoir; an air inducing device that provides an airflow over the wick to release the volatile substances to a surrounding environment; and a rotating assembly coupled to the wick, the rotating assembly configured to rotate the wick to periodically replenish the wick with the liquid mixture prior to introducing the wick to the airflow.

In another aspect, the disclosure is directed to a device for dispensing volatile substances to a surrounding environment, the device including a reservoir configured to hold a multi-component liquid mixture of non-volatile substances and volatile substances; a wick having a first portion and a second portion that are configured to be in selective communication with the mixture in reservoir; an air inducing device that provides an airflow over the first portion or the second portion of the wick to release the volatile substances to a surrounding environment; and a rotating assembly coupled to the wick, the rotating assembly configured to rotate so the wick cycles between a first position where the first portion of the wick is positioned to contact the mixture while the second portion of the wick is exposed to the airflow and a second position where the second portion of the wick is positioned to contact the liquid mixture while the first portion is exposed to the airflow.

In yet another aspect, the disclosure is directed to a device for dispensing volatile substances to a surrounding environment, the device including a reservoir configured to hold a multi-component liquid mixture of non-volatile substances and volatile substances; a wick having a first portion and a second portion that are configured to be in selective communication with the mixture in reservoir; and a rotating assembly coupled to the wick, the rotating assembly configured to rotate so the wick cycles between a first position where the first portion of the wick is positioned within the mixture while the second portion of the wick is exposed to the environment and a second position where the second portion of the wick is positioned within the liquid mixture while the first portion is exposed to the environment.

Other aspects of the invention will become apparent by consideration of the detailed description and accompanying drawings.

### BRIEF DESCRIPTION OF THE DRAWINGS

FIG. 1 is a schematic view of an embodiment of a device for dispensing volatile substances according to the present disclosure.

FIG. 2 is a perspective view of another example of an embodiment of a device for dispensing volatile substances according to the present disclosure.

FIG. 3 is a schematic side view of the device of FIG. 2.

FIG. 4 is a table of measured data illustrating a rate of mass loss of a volatile compound provided in a liquid mixture according to five different case conditions, the data demonstrating the effectiveness of the device illustrated in FIG. 2 in dispensing volatile substances.

FIG. 5 is a graph illustrating the amount of volatile substances and non-volatile substances remaining in the device in each of the five case conditions shown in FIG. 4.

FIG. 6 is a schematic side view of another example of an embodiment of a device for dispensing volatile substances according to the present disclosure.

FIG. 7 is a schematic top view of the device of FIG. 6.

FIG. 8 is a perspective view of a wick for use with the device of FIG. 6.

FIG. 9 is a schematic side view of another example of an embodiment of a device for dispensing volatile substances according to the present disclosure.

FIG. 10 is a schematic top view of the device of FIG. 9.

FIG. 11 is a perspective view of a wick for use with the device of FIG. 9.

### DETAILED DESCRIPTION

Before any embodiments of the disclosure are explained in detail, it is to be understood that the invention is not limited in its application to the details of construction and the arrangement of components set forth in the following description or illustrated in the following drawings. The disclosure is capable of other embodiments and of being practiced or of being carried out in various ways.

The concepts disclosed in this discussion are described and illustrated by referring to exemplary embodiments. These concepts, however, are not limited in their application to the details of construction and the arrangement of components in the illustrative embodiments and are capable of being practiced or being carried out in various other ways. The terminology in this document is used for the purpose of description and should not be regarded as limiting. Words such as “including,” “comprising,” and “having” and variations thereof as used herein are meant to encompass the items listed thereafter, equivalents thereof, as well as additional items.

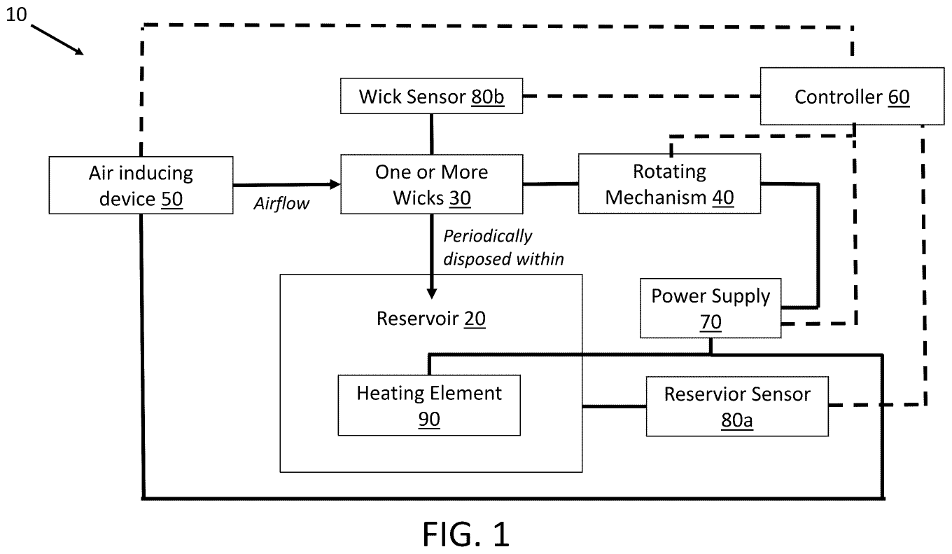

FIG. 1 schematically illustrates a device 10 for dispensing a volatile substance. Dispensing of a volatile substance during operation of the device 10 may deliver fragrances, incense, insect repellent, or other desirable volatile substances to a surrounding environment. It should be appreciated that the device 10 (or dispensing device 10) that is schematically illustrated in FIG. 1 is an example of a device provided to illustrate one or more potential (or optional) components of the device. However, in some embodiments, the device 10 may include any combination of the components schematically illustrated in FIG. 1.

With reference to FIG. 1, the device 10 includes a reservoir 20 configured to hold a multi-component liquid mixture of at least a non-volatile substance and a volatile substance. It should be appreciated that the multi-component liquid mixture can includes a plurality of volatile substances and/or a plurality of non-volatile substances. One or more wicks 30 are configured to be in selective communication with the liquid mixture in reservoir 20. The one or more wicks 30 are coupled to a rotation assembly 40. The rotating assembly 40 is configured to move the wicks 30 relative to the reservoir 20. More specifically, the rotating assembly 40 is configured to rotationally move the one or more wicks 30 relative to the assembly 40 into and out of selective contact with the liquid mixture in the reservoir 20. An air inducing device 50 is in fluid communication with the one or more wicks 30. The air inducing device 40 is configured to provide (or generate) an airflow. The airflow fluidly contacts the one or more wicks 30 after the at least one of the wicks 30 contacts the liquid mixture. Accordingly, the airflow generated by the air inducing device 40 is configured to release the volatile substance(s) from the one or more wicks 30 to the surrounding environment.

The device 10 can further include a controller 60 that is in communication with one or more components. In the illustrated embodiment, the controller 60 is in communication with the rotating assembly 40 and the air inducing device 50. The controller 60 can also be in communication with a power supply 70. The power supply 70 is configured to provide power to the rotating assembly 40, the air inducing device 50, and the controller 60. In some embodiments, the device 10 may further include one or more sensors 80a, 80b (e.g., a reservoir sensor 80a and a wick sensor 80b) and a heating element 90 in communication with the reservoir 20. The controller 60 can be in communication with the sensors 80a, 80b. The power supply 70 also provides power to the rotating assembly 40, the controller 60, and the heating element 90. The power supply 70 may be an internal power source (e.g., a battery) or an external power source (e.g., a wall plug or the like). It should be appreciated that FIG. 1 illustrates communication connections by broken lines.

The reservoir 20 may be formed of one or more different materials, such as plastic, glass, or any other suitable material configured to hold the multi-component mixture. In some embodiments, the reservoir 20 may be fully enclosed (e.g., have a lid) or have an opening to the surrounding environment. The multi-component mixture in the reservoir 20 includes a combination of one or more non-volatile substances and one or more volatile substances or volatile organic compounds (VOCs). As a non-limiting example, the non-volatile substance(s) can include Hexadecane and Dodecane, while the volatile substance(s) can include Decane. However, it should be appreciated that any suitable or desired non-volatile substance(s) and/or volatile substance(s) can be combined to form the multi-component mixture. As described in the example provided in the detailed experimental results below, the multi-compound mixture utilizes liquid solvents that are close to alkane hydrocarbons in properties. During operation of the device 10, the volatile substances are released from the liquid mixture and to the surrounding environment while the non-volatile substances remain in the liquid mixture. In some embodiments, diffuser oil formulations, insect repellent formulation, or the like may be developed with volatile substances for fragrances, insect repellent, or the like. The non-volatile substances may be odorless so they do not alter or obscure combined fragrances.

The one or more wicks 30 are formed of a porous material to facilitate the controlled absorption of the multi-component mixture in the reservoir and the controlled release of the volatile substances from the wicks 30. In some embodiments, the wicks 30 may be formed of materials such as a ceramic, polymer, clay, carbon, wood, cellulose fiber, fiberglass, or the like. As described in the example provided in the detailed experimental results below, the wicks 30 are formed of a polymer because the multi-component liquid mixture used in the experiment has a comparatively lower surface energy compared to the polymers (e.g., PE, PP, PC). The construction of the wicks 30 ensures the wettability of pore surfaces of polymer wicks 30 by the multi-compound mixture, and thus leading to spontaneous imbibition of the multi-compound mixture into the wick 30. However, it should be appreciated that the device 10 may be used with different multi-compound mixtures and materials of wicks to achieve desired absorption characteristics of the multi-compound mixture into the wicks and desired release of the volatile substances from the wicks. In the illustrated embodiment, the wicks 30 have a cylindrical or tubular shape.

The wicks 30 are coupled to the rotating assembly 40 such that the wicks are rotatable relative to the reservoir 20. The rotating assembly 40 rotates the wicks 30 such that the wicks 30 are cycled between a first position and a second position. In the first position, the at least one of the wicks 30 is disposed within (or into contact with) the mixture in the reservoir 20. In the second position, the at least one of the wicks 30 that was disposed within (or into contact with) the mixture in the first position is positioned within (or into contact with) the airflow induced by the air inducing device 50. The airflow dispenses the volatile liquids from the at least one of the wicks 30 to the surrounding environment. After exposure to the airflow, the rotating assembly 40 returns the at least one of the wicks 30 from the second position back to the first position to replenish the wicks 30 with the mixture from within the reservoir 20. This cycle then repeats to dispense the volatile substance from the mixture in the reservoir 20 into the surrounding environment. In addition, the rotating assembly 40 uses movement of the wicks 30 to stir the mixture in the reservoir 20, further improving the efficiency of the coating of the mixture on an outer surface of the wicks 30. When the wicks 30 are extracted from the reservoir 20, the airflow from air inducing device 50 is blown over the wicks 30 to dispense the volatile substances into the surrounding environment. The remaining lesser volatile part of the mixture on the wicks 30 is mixed back into the liquid mixture in the reservoir when the wicks 30 are submerged back into the reservoir 20. When the wicks 30 emerge from the reservoir 20 again, the wicks 30 are replenished with a new coating of the liquid mixture, which replenishes the volatile substance(s) on the wicks 30. In particular, the rotation assembly 40 allows an outer surface of the wick(s) 30 to be coated with the mixture each time the wick(s) 30 enter the reservoir 20, which reduces the amount of mixture that remains absorbed in the wick(s) 30. In other words, the rotation mechanism 40 makes it so the wicks 30 do not rely on capillary action of the mixture to expose the volatile substances of the mixture to the airflow.

The controller 60 is in communication with the rotating assembly 40 and can adjust the rotational speed of the wicks 30. In some embodiments, the controller 60 may continuously rotate the wicks 30 such that at least one wick is intermittently introduced to the airflow (in the second position) after the wick 30 is replenished with the mixture (in the first position). In some embodiments, the reservoir sensor 80a may detect the composition of the multi-compound mixture in the reservoir 20. The controller 60 may adjust the speed of the rotating assembly 40 to release the optimal amount of volatile substances into the surrounding environment based on composition detected by the reservoir sensor 80a. In some embodiments, the controller 60 may intermittently slow or stop rotation of the wicks 30 so one of the wicks 30 is positioned in the airflow. In such an embodiment, the wick sensor 80b may monitor the moisture of the wick 30 exposed to the airflow. In response to the wick sensor 80b detecting the wick 30 reaching a predetermined moisture level, the controller 60 may instruct the rotating assembly 40 to rotate the wicks 30 (or increase the speed of rotation of the wicks 30).

The air inducing device 50 provides an airflow over the wicks 30 to release the volatile substances to a surrounding environment. In some embodiments, the air inducing device 50 may be a fan coupled to the reservoir 20. In other embodiments, the air inducing device 50 may be another component or system, such as an HVAC system, configured to generate airflow and that the device 10 is positioned within or adjacent to. As such, it should be appreciated that the air inducing device 50 may be integral with the device 10 or formed and powered separately from the device 10.

The sensors 80a, 80b are in communication with the reservoir 20 to detect the composition of the multi-component mixture within the reservoir 20. For example, the reservoir sensor 80a may detect the amount of volatile substance(s) remaining in the multi-component mixture. Once the amount of volatile substances reaches a predetermined amount, the reservoir sensor 80b may send a signal to the controller 60. In response to the signal, the controller 60 may alert the operator of the device 10 that the multi-component mixture should be replaced with a fresh mixture (or additional volatile substance(s) should be added). In some embodiments, the wick sensor 80b may be in communication with the wicks 30 to detect one or more properties of the wicks 30. As a non-limiting example, the wick sensor 80b can detect a moisture content of the wicks 30, a weight (or change in weight) of the wicks 30, or any other suitable property that can be used to detect a changing amount of multi-component mixture in the wicks 30. In such embodiments, the controller 60 can responsively adjust the speed of the rotating assembly 40 in response to the properties of the wicks 30 detected by the wick sensor 80b. The heating element 90 is in communication with the reservoir 20 and is configured to heat the multi-component mixture. Raising the temperature of the multi-component mixture prior to coating the wick(s) 30 with the mixture can increases the rate that the volatile substances diffuse into the surrounding environment after being carried (or repositioned) into the airflow by the wick(s) 30.

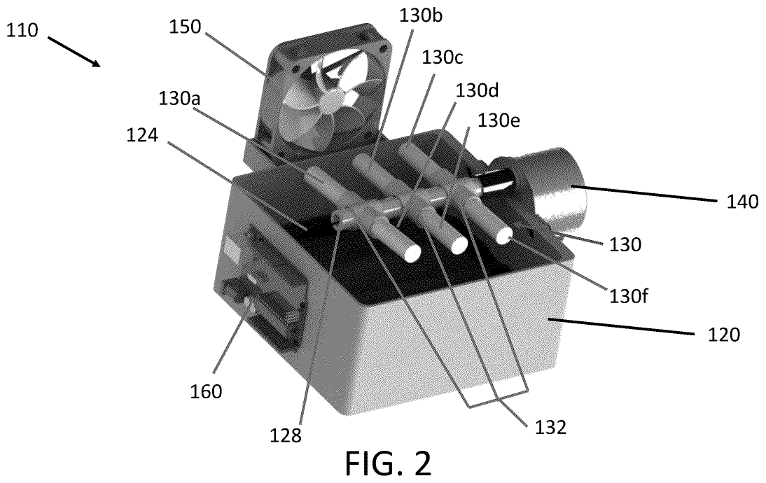

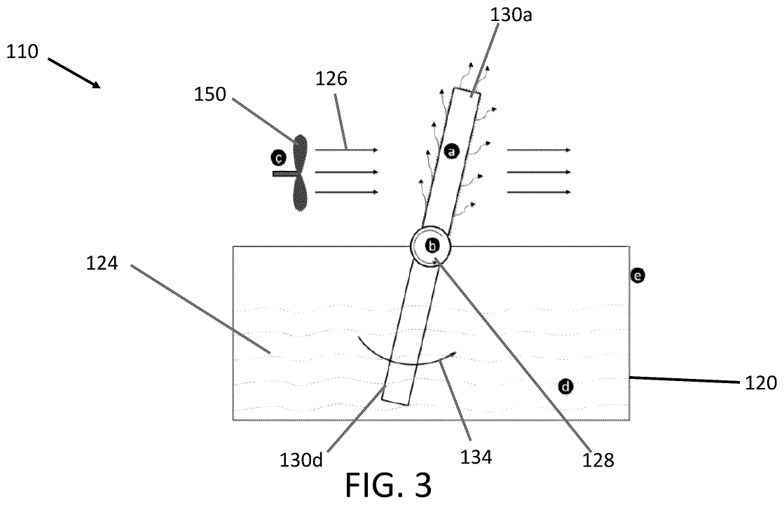

Now with reference to FIGS. 2 and 3, a device 110 of an example embodiment is illustrated. The device 110 is similar to the device 10 illustrated in FIG. 1. As such, like components are numbered with the same reference number plus “100”. It should also be appreciated that all of the components associated with device 10 and shown in FIG. 1 may be used with the device 110 illustrated in FIG. 2.

The device 110 includes a reservoir 120 configured to hold a multi-component liquid mixture 124 of at least a non-volatile substance and a volatile substance. A plurality of wicks 130 coupled to a support structure 128 (e.g., a shaft). A rotating assembly 140 (e.g., a stepper motor) is coupled to the reservoir 120. An air inducing device 150 (e.g., a fan) coupled to the reservoir 120 and provides an airflow 126 (FIG. 3) over one or more of the wicks 130 to release the volatile substances to a surrounding environment. A controller 160 is in communication with the rotating assembly 140 and the air inducing device 150. The support structure 128 is coupled to the rotating assembly 140 such that the rotating assembly 140 rotates the support structure 128 relative to the reservoir 120. The device 110 further includes wick holders 132 that are coupled to (or defined by) the support structure 128. The wick holders 130 are each configured to slidably receive one of the wicks 130.

In the illustrated embodiment, each wick holder 132 is supported on the support structure 128 and includes two receiving apertures that are respectively able to receive one of the plurality of wicks 130. In some embodiments, the apertures of each wick holder 132 may be continuous such that a single wick is slidably received through the apertures. Each wick 130 responsively extends on opposing sides of the support structure 128. In other embodiments, the wick holders 132 may be integrally formed with the support structure 128. In other examples of embodiments, the wick holders 132 can be circumferentially offset relative to the support structure 128. For example, each wick holder 132 can be radially offset from an adjacent wick holder 132 relative to the support structure 128. Thus, each wick 130 can be radially offset from an adjacent wick 130 relative to the support structure 128. Further, while the illustrated embodiment illustrates six total wicks 130a-f, it should be appreciated that the device 110 may include fewer wicks 130 (e.g., one, two, three, four, five) or more wicks 130 (e.g., seven, eight, etc.). In other examples of embodiments, the device 110 can include a plurality of wicks 130 or at least one wick 130.

The wick holders 132 are arranged such that a first set of wicks 130a-c are arranged on a first side of the support structure 128 and a second set of wicks 130d-f are arranged on a second side of the support structure 128. In other words, the first set of wicks 130a-c are positioned 180 degrees from the second set of wicks 130d-f. Stated another way, the first set of wicks 130a-c are generally parallel to the second set of wicks 130d-f. The orientation of the wicks 130a-f allows for the first set of wicks 130a-c to be exposed to the surrounding environment (and/or the associated airflow 126 generated by the air inducing device 150) while the second set of wicks 130d-f are being replenished with the multi-component mixture (shown in FIG. 3). It should be appreciated that in some embodiments the wicks 130a, 130d may be a single wick that extends through the support structure 128. In such an embodiment, the first set of wicks 130a-c define a first portion of the wicks 130 and the second set of wicks 130d-f define a second portion of the wicks 130.

Now with reference to FIG. 3, the device 110 is illustrated in operation. During operation, the rotation assembly 140 is configured to rotate the support structure 128 in a first direction, shown by arrow 136. The support structure 128 rotates relative to the reservoir 120 such that at least one wick 130a is exposed to the airflow 126 while at least one other wick 130d is submerged in the multi-component mixture 124 within the reservoir. The airflow 126 disperses the volatile substances on the wick 130a to the surrounding environment. As the support structure 128 continues to rotate, the wick 130a moves out of communication with the airflow 126 and into communication with the multi-component mixture 124 within the reservoir 120. Concurrently, the at least one other wick 130d rotates out of communication with the multi-component mixture 124 within the reservoir 120 and into communication with the airflow 126. This allows the wick 130a to replenish the liquid mixture on the wick 130a, while the other wick 130d is exposed to the airflow 126. As described in detail above, in other examples of embodiments, the controller 160 may adjust the rotational speed of the support structure 128. For example, the controller 160 may slow or intermittently stop rotation of the support structure 128 to effectively dispense the volatile substances from the wick(s) 130 positioned in the airflow 126 to the surrounding environment. Once the volatile substances are dispensed from the wick(s) 130, such as by detection with one or more of the sensors 80a, 80b (shown in FIG. 1), a predetermined amount of time, or other control step, the controller 160 may reinitiate rotation (or increase the speed of rotation) of the support structure 128. In other examples of embodiment, the air inducing device 150 can be optional, as the volatile material may not require additional or directed airflow to facilitate dispersion into the environment.

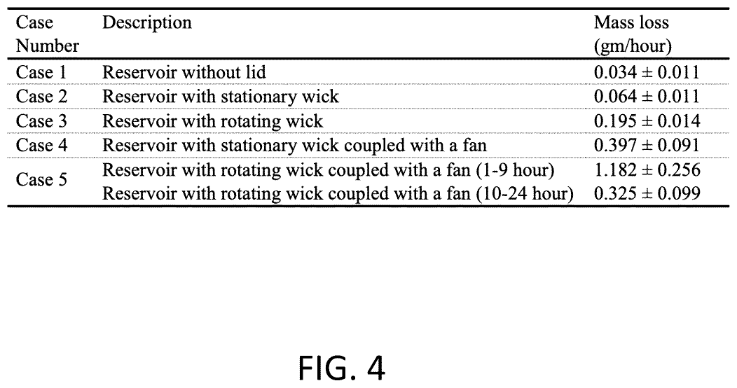

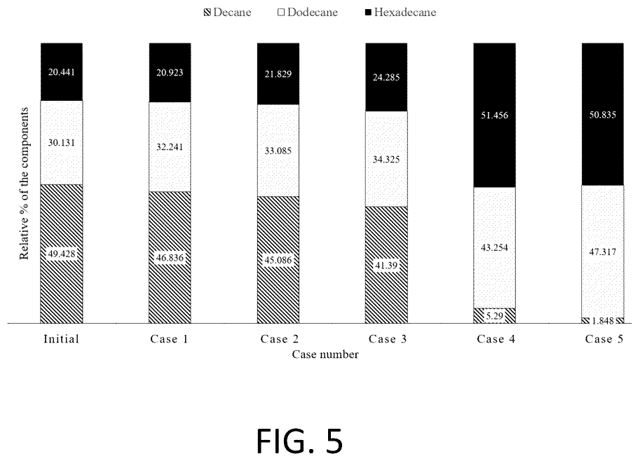

FIGS. 4 and 5 illustrate experimental data relating to the device 110 during operation. In particular, the experimental data for the device 110, which is provided in cases 4 and 5, illustrate the improved efficiency of dispersing volatile substances to the surrounding environment relative to other dispersing processes that do not include the device 110. The experimental data includes five (5) separate test conditions, referred to as “cases,” where the percentage of volatile and non-volatile substances in the multi-component liquid mixture were tested after a constant time period for each case, here twenty-four (24) hours. In each experimental condition, Decane was used to represent the volatile substance. Dodecane and Hexadecane were used to represent the non-volatile substance. Each test condition started with the same formulation of the multi-component liquid mixture (Decane, Dodecane, and Hexadecane). The multi-component liquid mixture was then analyzed after the twenty-four (24) hour period to determine a quantity of volatile (Decane) and non-volatile (Dodecane and Hexadecane) remaining in the multi-component liquid mixture. Further, a mass loss during the time period was also determined relative to an initial composition of the mixture. In the experiment, the initial composition includes 49.428% volatile substances (measured as a percentage of Decane) and 50.542% non-volatile substances (measured as a percentage of 30.131% Dodecane and 20.411% Hexadecane; see FIG. 5)

In case 1, the multi-component mixture is confined within a lidless reservoir in a calm, no-air-blown (or no air flow) environment. The mixture was introduced into the reservoir and was left undisturbed for a duration of 24 hours. When the liquid mixture is kept in an open reservoir, only the free surface area of the liquid is exposed to the surrounding air. As the mixture comes into direct contact with the air, the exposed surface area participates in evaporation, resulting in a steady loss of liquid mass over time. After 24 hours, the remaining mixture in the reservoir was analyzed to determine a reduction in mass and a relative ratio of remaining components, provided in a percentage. As shown in FIGS. 4 and 5, the mixture experienced a mass loss of 0.034±0.011 gram/hour over the 24 hour period (see FIG. 4) and the mixture had 46.836% volatile substances (measured as a percentage of Decane) remaining (see FIG. 5).

In case 2, the multi-component mixture is confined within a lidless reservoir with three stationary wicks in the reservoir. This arrangement is similar to a commercially available “plug-in” type air freshener. When the wicks are immersed in the reservoir, the wicks absorb the mixture due to capillary action. As the wick absorbs the liquid, the wicks pulls the mixture from the reservoir and transports it down the length of the wick by capillary action, while also exposing some liquid to the outside air as it wets the cylindrical surface of the wicks. After 24 hours, the remaining mixture in the reservoir was analyzed to determine a reduction in mass and a relative ratio of remaining components, provided in a percentage. The recorded mass loss for this scenario is measured to be 0.064±0.011 gram/hour over the 24 hour period (see FIG. 4), which is slightly higher than the mass loss in case 1. In addition, the mixture had 45.086% volatile substances remaining (measured as a percentage of Decane) (see FIG. 5), which is less than the amount of volatile substances remaining in case 1.

In case 3, the multi-component mixture confined within a lidless reservoir with three rotating wicks in the reservoir, but no added airflow was introduced. The rotation of the wicks stirs the liquid multi-component mixture continuously thus maintaining a uniform concentration ratio in the reservoir. In addition, rotation of the wicks replaces the liquid mixture on the wicks with a fresh coat of the liquid mixture, thereby restoring the concentration of volatile substances on the wick. Unlike case 2, rotation of the wicks spreads the mixture out across a larger area of the wicks, which lead to an increased rate of volatile evaporation. The higher concentration of the volatile substances on the outer surface of the wicks increases the evaporation rate of the volatile substances. In addition, no additional airflow was introduced. Accordingly, evaporation was driven solely by exposure to the surrounding environment. The recorded mass loss for this scenario is measured to be 0.195±0.014 gram/hour over the 24 hour period (see FIG. 4), which is three times higher than that in case 2. In addition, the mixture had 41.39% volatile substances remaining (measured as a percentage of Decane) (see FIG. 5), which is less than the amount of volatile substances remaining in cases 1 and 2.

In case 4, the multi-component mixture confined within a lidless reservoir with three stationary wicks in the reservoir and a fan providing an airflow over the wicks was measured. With the stationary wicks projecting outward from the multi-component mixture, the fan is employed to generate airflow over a portion of the wicks, notably the portion of the wicks exposed above the reservoir. The airflow created by the fan serves to enhance the evaporation process by forcing air over moist wick surfaces and thus promoting evaporation. This continuous airflow ensures that the air around the wick remains virtually devoid of evaporated liquid vapor, allowing for improved evaporation efficiency of the volatile component. The recorded mass loss for this scenario is measured to be 0.397±0.091 gram/hour, demonstrating a higher mass loss compared to cases 1-3. In addition, the mixture had 5.29% volatile substances remaining (measured as a percentage of Decane) (see FIG. 5), which is less than the amount of volatile substances remaining in cases 1-3.

In case 5, the multi-component mixture confined within a lidless reservoir with three rotating wicks in the reservoir and a fan providing an airflow over the wicks was measured (e.g., the illustrated device 110). The experimental data showed a distinct difference between the mass-loss histories of the first 9 hours (i.e., 1.182±0.256 gm/hour) compared to the subsequent experimental time (i.e., 0.325±0.099 gm/hour). The observed difference in mass loss rates between the initial 9 hours and the subsequent period signifies a distinct change in the evaporation dynamics over time. The data illustrates that the evaporation process undergoes a transition or stabilization phase after the first 9 hours of the experiment. The two distinct trendlines indicate that the evaporation rate in the latter period, spanning the last 14 hours, is significantly lower compared to the initial phase. The recorded mass loss for this scenario during the first 9 hours is measured to be 1.182±0.256 gram/hour and 0.325±0.099 gram/hour for the remaining 14 hours. As such, the experimental data illustrates that the illustrated device 110 efficiently increases the dispersion of volatile substances and a higher mass loss over at least the first 9 hours of the experiment compared to cases 1-4. In addition, the mixture had 1.848% volatile substances remaining (measured as a percentage of Decane) (see FIG. 5), which is less than the amount of volatile substances remaining in cases 1-4.

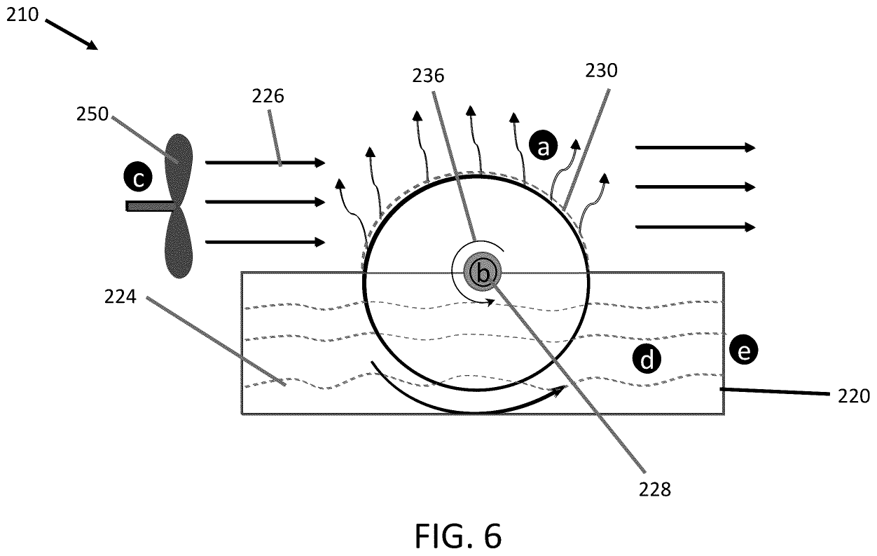

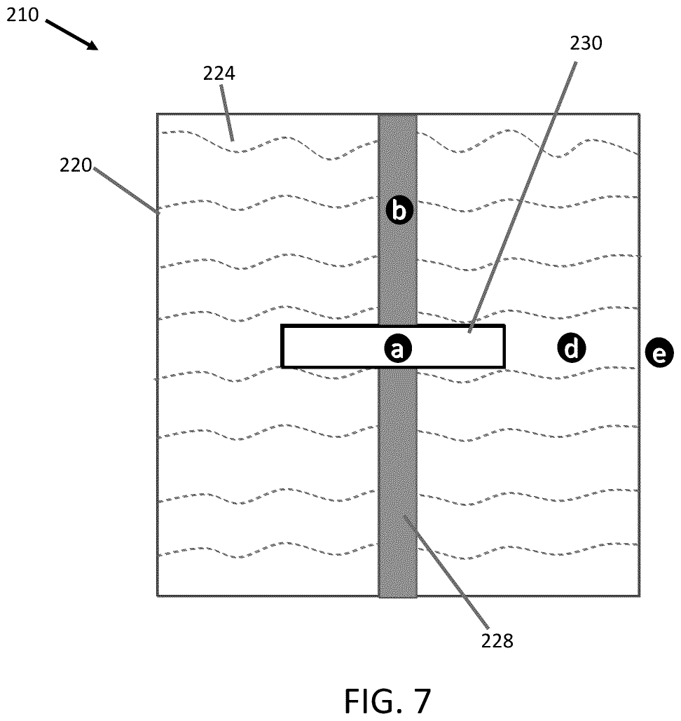

Now with reference to FIGS. 6-8, another example of an embodiment of device 210 is illustrated. The device 210 is similar to the device 10, 110 illustrated in FIGS. 1-3. As such, like components are numbered with the same reference number plus “200”. It should also be appreciated that all of the components associated with device 10 and device 110 and shown in FIGS. 1-3 may be used with the device 210 illustrated in FIGS. 6-8.

The device 210 includes a reservoir 220 configured to hold a multi-component liquid mixture 224 of at least a non-volatile substance and a volatile substance. A wick 230 coupled to a support structure 228 (e.g., a shaft). A rotating assembly (not shown, e.g., a stepper motor) is coupled to the support structure 228. An air inducing device 250 (e.g., a fan, an HVAC system, or the like) provides an airflow 226 (FIG. 6) to a portion of the wick 230 to release the volatile substances to a surrounding environment. The support structure 228 is coupled to the rotating assembly such that the rotating assembly rotates the support structure 228 relative to the reservoir 220.

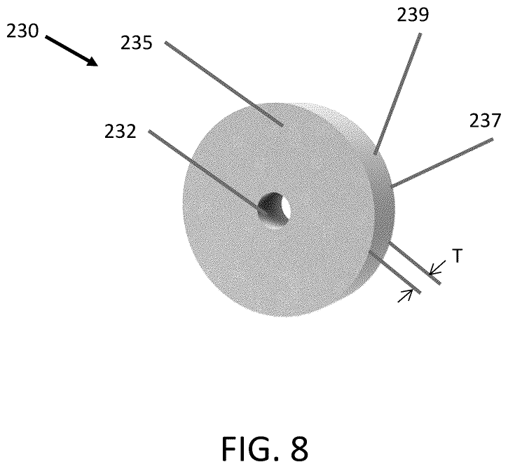

As illustrated in FIG. 8, the wick 230 includes an aperture 232 defined therein. The aperture 232 is configured to receive the support structure 228 to slidably support the wick 230. The wick 230 has a disk-shaped geometry having a first side 235, a second side 237, and an outer circumference 239 extending between the first and second sides 235, 237. Further, a thickness T is defined between the first and second sides 235, 237 of the wick 230. In the illustrated embodiment, the thickness T is in a range of approximately 1 millimeter to approximately 10 millimeters, and more specifically in a range of approximately 1 millimeter to approximately 4 millimeters. In other embodiments. In other examples of embodiments, the thickness T can greater than 1 millimeter, greater than 10 millimeters, or can be any suitable or desired thickness or range of thicknesses. In some embodiments, the support structure 228 may include a plurality of wicks with a similar geometry as the wick 230. Each of the plurality of wicks 230 can be spaced from an adjacent wick 230 along the support structure 228. It should be appreciated that the disk-shaped construction increases a surface area of the wick 230 that is continuously exposed to the airflow 226. This can advantageously increase the efficiency of the volatile substances dispersed from the wick 230 to the surrounding environment.

The wick 230 is arranged on the support structure 228 such that a first portion of the wick 230 is exposed to the surrounding environment (and/or the associated airflow 226 generated by the air inducing device 250) while a second portion of the wick 230 is being replenished with the multi-component mixture (shown in FIG. 6). Due to the continuous, disk-shaped construction of the wick 230, the wick 230 is in continuous contact with the mixture 224 in the reservoir 220 to continuously coat the sides 235, 237 and the outer circumference 239 of the wick 230 with the mixture.

With specific reference to FIG. 6, the device 210 is illustrated in operation. During operation, the rotation assembly is configured to rotate the support structure 228 in a first direction, shown by arrow 236. The support structure 228 rotates relative to the reservoir 220 such that the first portion of the wick 230 is exposed to the airflow 226 while the second portion of the wick 230 is submerged in the multi-component mixture 224 within the reservoir 220. The airflow 226 disperses the volatile substances on the first portion of the wick 230 to the surrounding environment. As the support structure 228 continues to rotate, the first portion of the wick 230 moves out of communication with the airflow 226 and into communication with the multi-component mixture 224 within the reservoir 220. Concurrently, the second portion of the wick 230 rotates out of communication with the multi-component mixture 224 within the reservoir 220 and into communication with the airflow 226. This allows a portion of the wick 230 to continuously be replenished, while the remaining portion of wick 230 is exposed to the airflow 226. As described in detail above, in other examples of embodiments, a controller may adjust the rotational speed of the support structure 228 to effectively dispense the volatile substances from the portion of the wick 230 positioned in the airflow 226 to the surrounding environment. Once the volatile substances are dispensed from the wick 230, such as by detection with one or more of the sensors 80a, 80b (shown in FIG. 1), by conclusion of a predetermined amount of time, or other suitable control step, the controller may reinitiate rotation (or change/increase the speed of rotation) of the support structure 228. In other examples of embodiment, the air inducing device 250 can be optional, as the volatile material may not require additional or directed airflow to facilitate dispersion into the environment.

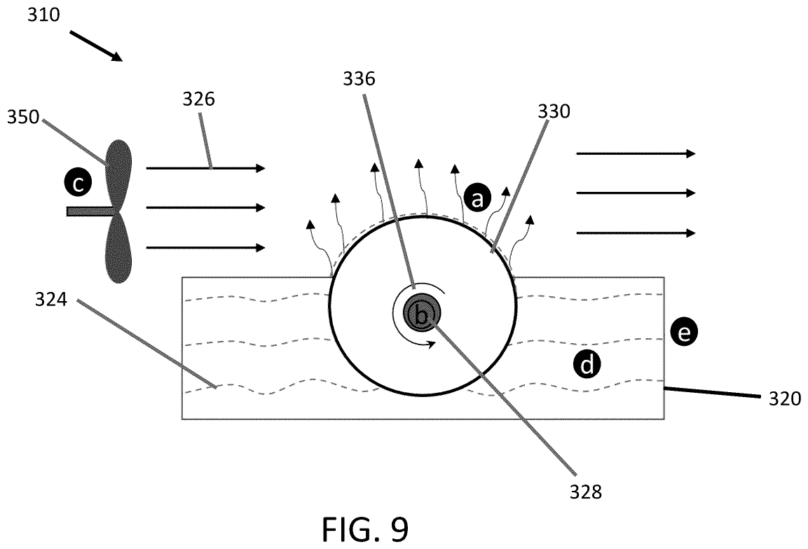

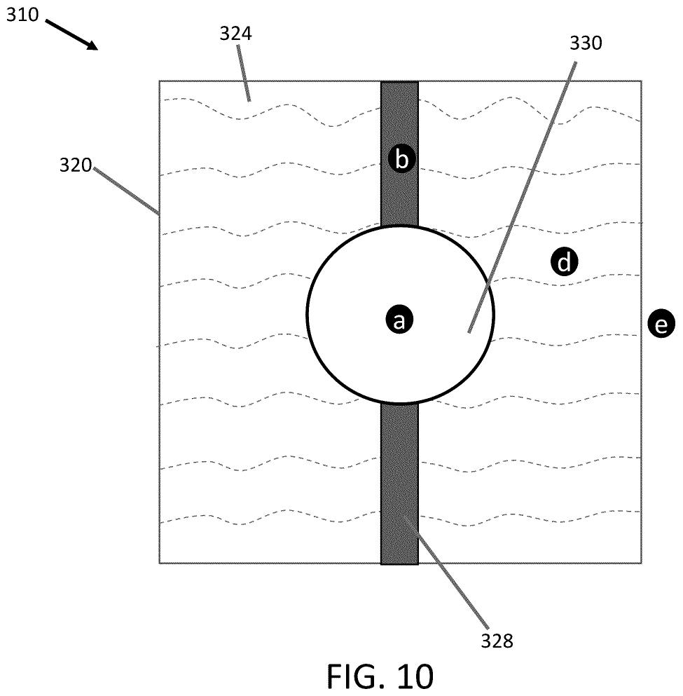

Now with reference to FIGS. 9-11, another example of an embodiment of a device 310 is illustrated. The device 310 is similar to the device 10, 110, 210 illustrated in FIGS. 1-3 and FIGS. 6-8. As such, like components are numbered with the same reference number plus “300”. It should also be appreciated that all of the components associated with device 10 and device 110 and shown in FIGS. 1-3 may be used with the device 310 illustrated in FIGS. 9-11.

The device 310 includes a reservoir 320 configured to hold a multi-component liquid mixture 324 of at least a non-volatile substance and a volatile substance. A wick 330 is coupled to a support structure 328 (e.g., a shaft). A rotating assembly (not shown, e.g., a stepper motor) is coupled to the support structure 328. An air inducing device 350 (e.g., a fan, HVAC system, or the like) provides an airflow 326 (FIG. 9) to a portion of the wick 330 to release the volatile substances to a surrounding environment. The support structure 328 is coupled to the rotating assembly such that the rotating assembly rotates the support structure 328 relative to the reservoir 320.

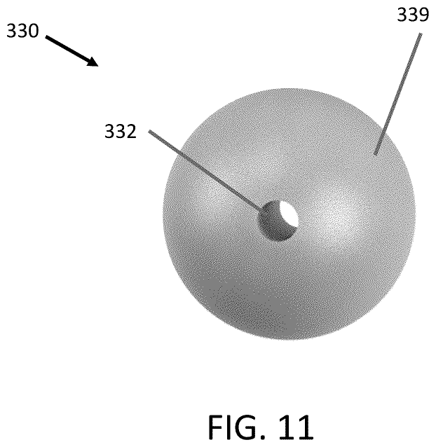

As illustrated in FIG. 11, the wick 330 includes an aperture 332 defined therein. The aperture 332 is configured to receive the support structure 328 to slidably support the wick 330. The wick 330 has a spherical, or generally ball-shaped, geometry defining an outer surface 339. In the illustrated embodiment, the wick 330 is a sphere with a constant diameter. However, it should be appreciated that the wick 330 may be a spheroid, an oblong sphere, or other spherical shape having different diameters or radii. In some embodiments, the support structure 328 may include a plurality of wicks with a similar geometry as the wick 330 such that each wick 330 is spaced offset from an adjacent wick 330 along the support structure 328.

The wick 330 is arranged on the support structure 328 such that a first portion of the wick 330 is exposed to the surrounding environment (and/or the associated airflow 326 generated by the air inducing device) while a second portion of the wick 330 is being replenished with the multi-component mixture (shown in FIG. 9). Due to the continuous, spherical construction of the wick 330, the wick 330 is in continuous contact with the mixture in the reservoir 320 to continuously coat the outer surface of the wick 330 with the mixture.

With specific reference to FIG. 9, the device 310 is illustrated in operation. During operation, the rotation assembly is configured to rotate the support structure 328 in a first direction, shown by arrow 336. The support structure 328 rotates relative to the reservoir 320 such that the first portion of the wick 330 is exposed to the airflow 326 while the second portion of the wick 330 is submerged in the multi-component mixture 324 within the reservoir 320. The airflow 326 disperses the volatile substances on the first portion of the wick 330 to the surrounding environment. As the support structure 328 continues to rotate, the first portion of the wick 330 moves out of communication with the airflow 326 and into communication with the multi-component mixture 324 within the reservoir 320. Concurrently, the second portion of the wick 330 rotates out of communication with the multi-component mixture 324 within the reservoir 320 and into communication with the airflow 326. This allows a portion of the wick 330 to continuously be replenished, while the remaining portion of wick 330 is exposed to the airflow 326. As described in detail above, in other examples of embodiments, a controller may adjust the rotational speed of the support structure 328 to effectively dispense the volatile substances from the portion of the wick 330 positioned in the airflow 326 to the surrounding environment. Once the volatile substances are dispensed from the wick 330, such as by detection with one or more of the sensors 80a, 80b (shown in FIG. 1), a predetermined amount of time, or other control step, the controller may reinitiate rotation (or change/increase the speed of rotation) of the support structure 328. In other examples of embodiment, the air inducing device 350 can be optional, as the volatile material may not require additional or directed airflow to facilitate dispersion into the environment.

Although the invention has been described in detail with reference to certain preferred embodiments, variations and modifications exist within the scope and spirit of one or more independent aspects of the invention as described.

Various features of the invention are set forth in the following claims.

What is claimed is:

1. A device for dispensing volatile substances to a surrounding environment, the device comprising
a reservoir configured to hold a multi-component liquid mixture of non-volatile substances and volatile substances;
a wick configured to be in selective communication with the liquid mixture in reservoir;
an air inducing device that provides an airflow over the wick to release the volatile substances to a surrounding environment; and
a rotating assembly coupled to the wick, the rotating assembly configured to rotate the wick to periodically replenish the wick with the liquid mixture prior to introducing the wick to the airflow.

2. The device of claim 1, further comprising a support structure coupled to the rotating assembly, wherein the wick is coupled to the support structure, and wherein the rotating assembly rotates the support structure relative to the reservoir.

3. The device of claim 2, further comprising a wick holder coupled to the support structure, and wherein the wick holder is configured to slidably receive the wick.

4. The device of claim 1, further comprising a controller in communication with the rotating assembly and a power supply configured to provide power to the rotating assembly.

5. The device of claim 4, further comprising a sensor in communication with the reservoir, and wherein the sensor is configured to detect properties of the multi-component mixture within the reservoir.

6. The device of claim 5, wherein the sensor is configured to detect a composition of the multi-component liquid mixture within the reservoir, and wherein the controller is configured to alert an operator of the device that the multi-component mixture should be replaced with a fresh mixture when an amount of the volatile substances in the composition of the multi-component liquid mixture reaches a predetermined amount.

7. A device for dispensing volatile substances to a surrounding environment, the device comprising
a reservoir configured to hold a multi-component liquid mixture of non-volatile substances and volatile substances;
a wick having a first portion and a second portion that are configured to be in selective communication with the mixture in reservoir;
an air inducing device that provides an airflow over the first portion or the second portion of the wick to release the volatile substances to a surrounding environment; and
a rotating assembly coupled to the wick, the rotating assembly configured to rotate so the wick cycles between a first position where the first portion of the wick is positioned to contact the mixture while the second portion of the wick is exposed to the airflow and a second position where the second portion of the wick is positioned to contact the liquid mixture while the first portion is exposed to the airflow.

8. The device of claim 7, wherein the wick is formed of a single piece defining the first and second portions.

9. The device of claim 7, wherein the wick includes a first wick defining the first portion and a second wick defining the second portion.

10. The device of claim 7, further comprising a support structure coupled to the rotating assembly, wherein the wick is coupled to the support structure, and wherein the rotating assembly rotates the support structure relative to the reservoir.

11. The device of claim 10, further comprising a wick holder coupled to the support structure, and wherein the wick holder is configured to slidably receive the wick.

12. The device of claim 7, further comprising a controller in communication with the rotating assembly and a power supply configured to provide power to the rotating assembly.

13. The device of claim 12, further comprising a sensor in communication with the reservoir, and wherein the sensor is configured to detect properties of the multi-component mixture within the reservoir.

14. The device of claim 13, wherein the sensor is configured to detect a composition of the multi-component liquid mixture within the reservoir, and wherein the controller is configured to alert an operator of the device that the multi-component mixture should be replaced with a fresh mixture when an amount of the volatile substances in the composition of the multi-component liquid mixture reaches a predetermined amount.

15. A device for dispensing volatile substances to a surrounding environment, the device comprising
a reservoir configured to hold a multi-component liquid mixture of non-volatile substances and volatile substances;
a wick having a first portion and a second portion that are configured to be in selective communication with the mixture in reservoir; and
a rotating assembly coupled to the wick, the rotating assembly configured to rotate so the wick cycles between a first position where the first portion of the wick is positioned within the mixture while the second portion of the wick is exposed to the environment and a second position where the second portion of the wick is positioned within the liquid mixture while the first portion is exposed to the environment.

16. The device of claim 15, further comprising a support structure coupled to the rotating assembly, wherein the wick is coupled to the support structure, and wherein the rotating assembly rotates the support structure relative to the reservoir.

17. The device of claim 16, further comprising a wick holder coupled to the support structure, and wherein the wick holder is configured to slidably receive the wick.

18. The device of claim 15, further comprising a controller in communication with the rotating assembly and a power supply configured to provide power to the rotating assembly.

19. The device of claim 15, wherein the wick is disc shaped, spherical in shape, or tubular in shape.

20. The device of claim 19, wherein the sensor is configured to detect a composition of the multi-component liquid mixture within the reservoir, and wherein the controller is configured to alert an operator of the device that the multi-component mixture should be replaced with a fresh mixture when an amount of the volatile substances in the composition of the multi-component liquid mixture reaches a predetermined amount.
